(19) United States
(12) Patent Application Publication
(10) Pub. No.: US 2014/0099353 A1
(43) Pub. Date: Apr. 10, 2014

(54) METHOD FOR PRODUCING AN IMPLANT COATING, AND CORRESPONDING IMPLANT

(71) Applicant: Helmut D. Link, Hamburg (DE)

(72) Inventor: Helmut D. Link, Hamburg (DE)

(73) Assignee: DERU GmbH, Norderstedt (DE)

(21) Appl. No.: 14/123,711

(22) PCT Filed: May 30, 2012
(86) PCT No.: PCT/EP2012/060110

§ 371 Date: Dec. 3, 2013

(30) Foreign Application Priority Data
Jun. 3, 2011 (EP) 11 168 635.8

(51) Int. Cl. A61L 27/54; A61L 27/30; A61L 27/32

(52) U.S. Cl. CPC A61L 27/54; A61L 27/32; A61L 27/306
USPC 424/423; 424/602; 424/687; 427/2.26; 427/2.27

(57) ABSTRACT

A medical implant which, on at least part of its surface, has a coating with an osteoinductive and/or osteoconductive top layer based on calcium phosphate and/or calcium carbonate, wherein an antibiotic active substance, which is soluble in aqueous medium, is coated over the osteoinductive and/or osteoconductive top layer in patches, leaving gaps on the osteoinductive and/or osteoconductive top layer.

### REFERENCE TO RELATED APPLICATIONS

This application is a national stage application under 35 USC 371 of International Application No. PCT/EP2012/060110, filed May 30, 2012, which claims priority to European application no. 11168635.8, filed Jun. 3, 2011, the entire contents of which are incorporated herein by reference.

### FIELD OF THE INVENTION

The invention relates to a method for producing an implant coating and to a corresponding implant. The implants are intended to be used in human and veterinary medicine for treating bone defects and encompass both temporary and permanent implants. The implant can, for example, be a prosthesis which remains in the body, known as an endoprosthesis.

### BACKGROUND OF THE INVENTION

Each medical implant constitutes a foreign body for the implantee and therefore brings about a complex biological interaction on a very wide variety of different levels. One of the most important reactions of the body is recruitment of osteogenic stem cells to the implant surface, known as osteoconduction. In this process, in a first step, the implant surface absorbs fibrinogen, to which there is attachment of platelets, which on their part release osteogenic growth factors when activated and induce migration of osteogenic stem cells to the implant, specifically the implant surface. The osteogenic stem cells secrete an organic bone matrix, which is mineralized by calcium phosphate deposition. In the ideal case, the implant is tightly joined to the bone following completed osteoconduction, which imparts primary stability, and osteointegration, which imparts secondary stability.

It is known that the roughness of the implant surface affects the process of osteoconduction, with increasing roughness, for example by coating of the implant by means of a thin calcium phosphate layer, being associated with better osteointegration. Initial experimental results on animals show improved osteointegration of calcium phosphate-coated implants compared to uncoated control implants (Junker et al., Effects of implant surface coatings and composition on bone integration: a systematic review. Clinical Oral Implants Research, Volume 20 Issue Supplement 4: 185-206, September 2009). Apparently, the calcium phosphate acts both osteoconductively and osteoinductively, i.e., it firstly serves as support structure for the osteoblasts and secondly promotes new bone formation, i.e., the engraftment of the implant on the bone. Furthermore, the calcium phosphate coat masks the artificial implant, and so it is no longer recognized as a foreign body.

From clinical practice, it is likewise known that any implant is a preferred substratum for the colonization of bacteria. Certain bacteria such as Staphylococcus aureus are capable of forming on the implant a biofilm composed of extracellular mucus, in which bacterial microcolonies form and multiply until the biofilm has covered the entire implant. With increasing bacterial infection, which can extend over years and is frequently associated with loosening of the implant, systemic treatment with antibiotics is successful in very rare cases, since the biofilm forms a “protective wall” for the bacterial colonies, and behind said wall, therapeutically effective antibiotic concentrations do not appear, even in the case of high-dose systemic administration of antibiotics. Also, systemically administered antibiotics barely reach the surroundings of the implant, since the tissue on the implant is frequently cicatrized and thus poorly supplied with blood. As a result, surgical restoration needs to take place, i.e., the implant has to be removed and the bacterial infection treated locally.

To ensure high effective antibiotic levels on the implant which prevent colonization by bacteria and, more particularly, biofilm formation and to counteract possible subsequent bacterial infection, it is useful to coat the implant with antibiotics. From the prior art, it is known to apply antibiotics by means of a binder or by embedding in an organic matrix on the porous surface of the implant as a layer, with the implant being overcoated over the entire surface (Moskowitz et al., The effectiveness of the controlled release of gentamicin from polyelectrolyte multilayer in the treatment of Staphylococcus aureus infection in rabbit bone model, Biomaterials (2010) volume 31, issue 23: 6019-6030, August 2010; Vester et al., Gentamycin delivered from a PDLLA coating of metallic implants In vivo and in vitro characterisation for local prophylaxis of implant-related osteomyelitis, Injury 2010: 1053-1059; DE 10 2005 002 703).

### SUMMARY OF THE INVENTION

It is an object of the invention to provide medical implants which become incorporated in the body with virtually no infection and with the formation of a tight join to the bone.

More particularly, it is an object of the invention to develop an antibiotic implant coating which, firstly, releases effective amounts of antibiotics locally at the interface between implant and the tissue from a corresponding antibiotic coating (aim 1), but, secondly, does not deny access to the calcium phosphate- and/or calcium carbonate-based cover layer which lies under the antibiotic coating and acts osteoinductively and osteoconductively (aim 2).

This conflict between the aims, which conflict is not possible to resolve at first sight, is solved, surprisingly, by the solution according to the invention in the features as broadly described herein. In the various detailed embodiments described below, advantageous and appropriate further developments are specified.

A method for producing a coating on a medical implant and a corresponding implant are envisaged, comprising the coating of at least part of the surface of the medical implant with an osteoinductive and/or osteoconductive cover layer based on calcium phosphate and/or calcium carbonate, wherein, according to the invention, it is provided that an active antibiotic ingredient which is soluble in an aqueous environment overcoats the osteoinductive and/or osteoconductive layer in such a manner that patches are formed with spaces being left free on the osteoinductive and/or osteoconductive cover layer.

The method according to the invention and the implant produced according to said method make it possible to avoid the aforementioned conflict between the aims and to fully combine the advantages of an antibiotic layer with those of an osteoinductive and/or osteoconductive cover layer based on calcium phosphate and/or calcium carbonate, leading, as a result, to infection-free incorporation of the implant in the body with the formation of a tight join between implant and bone.

In the context of the present invention, the patchy overcoating of the osteoinductive and/or osteoconductive cover layer means that the cover layer is provided with sites to which the active antibiotic ingredient or the active ingredient-bearing layer or matrix is applied. Accordingly, the gaps between the patches do not have any active antibiotic ingredient. For the purposes of the invention, the patchy overcoating consists of a group of individual patches or gaps of varying dimensions and shapes. Preferably, the patches containing the active antibiotic ingredient cover between 1% and 95%, preferably between 5% and 90%, more preferably between 10% and 85%, more preferably between 15% and 80%, more preferably between 20% and 75%, more preferably between 25% and 75%, more preferably between 30% and 70%, more preferably between 35% and 65%, more preferably between 40% and 60%, more preferably between 45% and 55%, more preferably about 50% of the osteoinductive and/or osteoconductive cover layer lying therebelow. The size of the patches can vary, wherein a drop-shaped impact on the osteoinductive and/or osteoconductive cover layer preferably produces patches having a diameter of 0.5-20 mm, 0.5-15 mm, 0.5-10 mm, 0.5-5 mm, 0.5-4 mm, 0.5-3 mm, 0.5-2 mm, 0.5-1 mm, 1-20 mm, 1-15 mm, 1-10 mm, 1-5 mm, 1-4 mm, 1-3 mm, 1-2 mm. The mean patch diameter can be in a range of 0.75-20 mm, 1-20 mm, 2-15 mm, 3-10 mm and 4-5 mm. The aforementioned parameters of surface coverage and patch sizes can be combined with one another.

In a preferred embodiment of the invention, the osteoinductive and/or osteoconductive cover layer based on calcium phosphate can comprise hydroxylapatite. Hydroxylapatite is a resorbable biomaterial which has already frequently proved itself in practice as bone substitute material and is, in this regard, predominantly used as coating material to make use of the advantages of its osteoinductive and osteoconductive action. However, it is also possible to use other calcium phosphate layers, for example α- and/or β-tricalcium phosphate, tetracalcium phosphate or mixtures of these variants, optionally with calcium oxide additives.

In principle, any active antibiotic ingredient which develops its antibacterial action under in vivo application conditions, i.e., especially at body temperature and in an aqueous environment, is possible in the context of the present invention. In medical practice, the control of bacterial infections has proven successful with especially aminoglycoside antibiotics, preferably gentamicin and amikacin, but also apramycin, geneticin (G418), kanamycin, netilmicin, neomycin, paromomycin, spectinomycin, streptomycin, tobramycin; lincosamide antibiotics, preferably clindamycin, lincomycin; cephalosporin antibiotics, preferably cefuroxime and cefoperazone; fluoroquinolone antibiotics, preferably ofloxacin; glycopeptide antibiotics, preferably vancomycin; β-lactam antibiotics, preferably ampicillin and the corresponding salts thereof.

Of particular practical relevance is the aminoglycoside antibiotic gentamicin, which counteracts the Staphylococcus aureus strains which are particularly significant for infections and which are especially also substantially involved in the formation of the “protective wall” biofilm, as already described at the start. However, since even the antibiotic activity spectrum of gentamicin has gaps and there is especially the risk of acquired gentamicin resistance, it is advantageous to supplement gentamicin with further antibiotics. Resistances against frequently used antibiotics can be regularly found especially in the hospital sector and can be successfully controlled in many cases only by combining multiple antibiotics having different mechanisms of action. For example, gentamicin can be combined with the lincosamide antibiotic clindamycin in order to act synergistically against staphylococci, streptococci and propionibacteria. A similar spectrum of activity is exhibited by the combination of gentamicin and the cephalosporin antibiotic cefuroxime. To prevent Pseudomans infections, use can be made of a combination of gentamicin, fluoroquinolone antibiotics ofloxacin or cefoperazone and further aminoglycoside antibiotics amikacin. Of particular relevance in clinical practice are also the methicillin-resistant Staphylococcus aureus (MRSA) and methicillin-resistant Staphylococcus epidermidis (MRSE) strains, which now increasingly appear in hospitals and which can be controlled with a chance of success using a combination of gentamicin, the glycopeptide antibiotic vancomycin and the fluoroquinolone antibiotic ofloxacin. In the case of an Enterococcus infection and to combat vancomycin resistances, a combination of vancomycin, gentamicin and the β-lactam antibiotic ampicillin is useful. Irrespective of the use of antibiotics on the implant according to the invention, it may also be advantageous to carry out an accompanying systemic antibiotic therapy in a preventative manner and/or after the implantation of the implant.

In a preferred embodiment of the invention, the antibiotics or the salts thereof adhere by themselves or via a support, preferably a (e.g., polymeric) layer former, or by embedding in a matrix to the osteoinductive and/or osteoconductive layer. The support or the matrix formers can, for example, be synthesized from stearic acid, palmitic acid, myristic acid, behenic acid, myristyl palmitate, cetyl palmitate or ceryl cerotinate, which adhere well to metal and plastic surfaces.

In the context of the invention, all antibiotic salts are possible, including water-soluble salts of gentamicin, of sisomicin, of netilmicin, of streptomycin, of tobramycin, of spectinomycin, of vancomycin, of ciprofloxacin, of moxifloxacin, of clindamycin, of lincomycin, of tetracycline, of chlortetracycline, of oxytetracycline and of rolitetracycline, with preference being given to gentamicin salts of palmitic acid, of lauric acid, of stearic acid, of oleic acid, of phenylbutyric acid, of naphthalene-1-carboxylic acid or sulfates of gentamicin.

It may be advantageous to ensure the desired retarding release of active ingredient by means of poor or slight solubility of the active antibiotic ingredient in an aqueous environment, for example by using antibiotic salts which are slightly or poorly soluble in an aqueous environment. Examples include the antibiotic salts from the group of netilmicin laurate, netilmicin dodecyl sulfate, netilmicin myristate, sisomicin laurate, sisomicin myristate, sisomicin dodecyl sulfate, gentamicin laurate, gentamicin myristate, clindamycin laurate, amikacin laurate, amikacin myristate, amikacin dodecyl sulfate, kanamycin laurate, kanamycin myristate, kanamycin dodecyl sulfate, vancomycin laurate, vancomycin dodecyl sulfate, vancomycin myristate, vancomycin-teicoplanin, tobramycin laurate, tobramycin myristate, tobramycin dodecyl sulfate, ciprofloxacin laurate, ciprofloxacin myristate, clindamycin-teicoplanin, fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlortetracycline, fusidic acid-oxytetracycline and fusidic acid-rolitetracycline. The poorly soluble salts can be dissolved in appropriate organic solvents and applied with these solutions, optionally with the addition of a layer former or of an embedding matrix, to the osteoinductive and/or osteoconductive cover layer.

1. A medical implant having on at least part of its surface a coating having one or both of an osteoinductive and an osteoconductive cover layer based on one or both of calcium phosphate and calcium carbonate wherein an active antibiotic ingredient which is soluble in an aqueous environment overcoats the one or both of the osteoinductive and the osteoconductive cover layer in a patchy manner with spaces being left free on the one or both of the osteoinductive and the osteoconductive cover layer.

2. The medical implant of claim 1, wherein the one or both of the osteoinductive and the osteoconductive cover layer based on calcium phosphate comprises hydroxylapatite.

3. The medical implant of claim 1, wherein the active antibiotic ingredient comprises at least one antibiotic selected from the group consisting of:
aminoglycoside antibiotics,
lincosamide antibiotics,
cephalosporin antibiotics,
fluoroquinolone antibiotics,
glycopeptide antibiotics, and
β-lactam antibiotics or the salts thereof.

4. The medical implant of claim 3, wherein the antibiotic or the salts thereof adhere directly or via a support on the one or both of the osteoinductive and the osteoconductive layer.

5. The medical implant of claim 3, wherein the antibiotic salts are gentamicin salts of palmitic acid, of lauric acid, of stearic acid, of oleic acid, of phenylbutyric acid, of naphthalene-l-carboxylic acid or sulfates of gentamicin.

6. The medical implant of claim 1, wherein the active antibiotic ingredient is slightly or poorly soluble in an aqueous environment.

7. The medical implant of claim 1, wherein the patchy overcoating of the one or both of the osteoinductive and the osteoconductive layer with the active antibiotic ingredient which is soluble in an aqueous environment is achieved by in situ application with spaces being left free on the one or both of the osteoinductive and the osteoconductive layer.

8. A method for producing a coating on a medical implant, comprising:
coating at least part of the surface of the medical implant with one or both of an osteoinductive and an osteoconductive cover layer based on one or both of calcium phosphate and calcium carbonate, using an active antibiotic ingredient which is soluble in an aqueous environment to overcoat the one or both of the osteoinductive and the osteoconductive layer so that patches are formed with spaces being left free on the one or both of the osteoinductive and the osteoconductive cover layer.

9. The method of claim 8, wherein the one or both of the osteoinductive and the osteoconductive cover layer based on calcium phosphate comprises hydroxylapatite.

10. The method of claim 8, wherein the active antibiotic ingredient comprises at least one antibiotic selected from the group consisting of:
aminoglycoside antibiotics,
lincosamide antibiotics,
cephalosporin antibiotics,
fluoroquinolone antibiotics,
glycopeptide antibiotics, and
β-lactam antibiotics, or the salts thereof.

11. The method of claim 10, wherein the antibiotic or the salts thereof adhere directly or via a support on the one or both of the osteoinductive and the osteoconductive layer.

12. The method of claim 10, wherein the antibiotic salts are gentamicin salts of palmitic acid, of lauric acid, of stearic acid, of oleic acid, of phenylbutyric acid, of naphthalene-1-carboxylic acid or sulfates of gentamicin.

13. The method of claim 8, wherein the active antibiotic ingredient is slightly or poorly soluble in an aqueous environment.

14. The method of claim 8, wherein the patchy overcoating of the one or both of the osteoinductive and the osteoconductive layer with the active antibiotic ingredient which is soluble in an aqueous environment is achieved by in situ application with spaces being left free on the one or both of the osteoinductive and the osteoconductive layer.

15. A medicinal implant obtainable by a method of claim 8.

16. The medical implant of claim 3, wherein the aminoglycoside antibiotics comprise gentamicin and amikacin, the lincosamide antibiotics comprise clindamycin and lincomycin, the cephalosporin antibiotics comprise cefuroxime and cefoperazone, the fluoroquinolone antibiotics comprise ofloxacin, the glycopeptide antibiotics comprise vancomycin, and the β-lactam antibiotics comprise ampicillin.

17. The medical implant of claim 4, wherein the support comprises a polymeric layer former.

18. The medical implant of claim 7, wherein the in situ application comprises spraying, drop application or pipetting of a solution or suspension containing the active antibiotic ingredient.

19. The method of claim 10, wherein the aminoglycoside antibiotics comprise gentamicin and amikacin, the lincosamide antibiotics comprise clindamycin and lincomycin, the cephalosporin antibiotics comprise cefuroxime and cefoperazone, the fluoroquinolone antibiotics comprise ofloxacin, the glycopeptide antibiotics comprise vancomycin, and the β-lactam antibiotics comprise ampicillin.

20. The method of claim 11, wherein the support comprises a polymeric layer former.

21. The method of claim 14, wherein the in situ application comprises spraying, drop application or pipetting of a solution or suspension containing the active antibiotic ingredient.
